(19) United States
(12) Patent Application Publication
(10) Pub. No.: US 2023/0302251 A1
(43) Pub. Date: Sep. 28, 2023

(54) METHOD TO ACCELERATE SLEEP ONSET AND/OR TO IMPROVE SLEEP QUALITY IN A SUBJECT

(71) Applicant: Metaphysiks Engineering SA, Nyon (CH)

(72) Inventors: Giulio ROGNINI, Zurich (CH); Simon GALLO, Arzier (CH); Olaf BLANKE, Nyon (CH)

(21) Appl. No.: 18/004,312

(22) PCT Filed: Jul. 9, 2021
(86) PCT No.: PCT/EP2021/069230

§ 371 Date: Jan. 5, 2023

(30) Foreign Application Priority Data
Jul. 10, 2020 (EP) 20020319.8

(51) Int. Cl. A61M 21/00; G16H 20/30

(52) U.S. Cl. CPC A61M 21/00; G16H 20/30; A61M 2021/0022; A61M 2021/0027; A61M 2021/005; A61M 2021/0066; A61M 2205/0216; A61M 2205/3303; A61M 2205/3344; A61M 2205/3368; A61M 2205/3569; A61M 2230/50

(57) ABSTRACT

Methods, devices and systems to accelerate sleep onset and/or to improve sleep quality in a subject are herein disclosed.

### TECHNICAL FIELD

The invention lies in the fields of methods, devices and systems to accelerate sleep onset and/or to improve sleep quality in a subje ct.

### BACKGROUND ART

People spend more time sleeping than on any other single activity throughout their lives. Sleep is rapidly becoming a fundamental component of the third pillar of well-being and is poised to undergo the same vast transformation that fitness and nutrition have as they became major consumer categories.

Sleep insufficiency causes high human costs with major impact on the physical and mental health of affected individuals with economic costs, mostly resulting from loss in productivity. For example, sleep insufficiencies in older adults are often associated with deficits in daytime functioning including elevated levels of fatigue, disturbed cognitive performance and mood, and clinical insomnia. Yet, despite many ground-breaking discoveries about the intricate physiology of sleep, the definition of sleep stages, and dream research since the 1950s, sleep insufficiency and related impairments continue to grow in today’s society. This is likely related to the complex nature and many factors influencing sleep, including life style changes and an aging population.

Classical solutions for sleep problems, such as insomnia, often rely on pharmacological treatments. Yet, such treatments provide only temporary remediation, and have side effects including dependency. Non-pharmacological solutions, such as cognitive behaviour therapy, focus on modulating sleep needs and correcting expectations, attitudes, and beliefs about sleep. Differing from pharmacotherapy, these latter therapies have more long-terms effect on sleep quality and have no serious contraindications. However, they require administration by highly trained therapists, making them expensive and in many regions and circumstances unavailable.

Thermoregulation and its circadian pattern have recently been shown to influence several aspects of sleep. Thus, experimental studies in humans have highlighted the importance of temperature changes during sleep onset by showing that ‘therapeutic’ thermal stimulation of a person’s extremities (i.e. hands and feet) accelerates sleep onset (Kräuchi, Kurt & Cajochen, Christian & Werth, Esther & Wirz-Justice, Anna. (1999). Warm feet promote the rapid onset of sleep. Nature. 401. 36-37. 10.1038/43366). Despite the importance of thermoregulation for sleep, there is currently no solution that attempts to engineer the bodily thermal changes physiologically required by the process of falling asleep.

Among non-pharmacological interventions, mind-body interventions such as relaxation-meditation techniques have been extensively studied in sleep research and shown to accelerate sleep onset and improve sleep quality. For instance, relaxation-mediation can attenuate disturbed mood and distress (i.e. increase of attentional control of autonomic nervous system, reduction of worry and rumination, attenuate mood disturbances) (Neuendorf, Rachel et al. “The Effects of Mind-Body Interventions on Sleep Quality: A Systematic Review.” Evidence-based complementary and alternative medicine: eCAM vol. 2015 (2015): 902708.). However, it is currently unknown whether and how thermoregulation could be combined with relaxation-meditation, beyond off-the-shelf solutions such as foot-bath devices commonly used at home. The latter have very poor temporal and spatial control over foot temperature regulation and it is not possible to integrate them with the precise spatio-temporal features necessary for relaxation-meditation scenarios.

Accordingly, the psychological, physiological, and neural mechanisms underlying pre-sleep foot thermoregulation and pre-sleep meditation/relaxation techniques to improve sleep remain poorly understood and have never been systematically investigated. The integration of both approaches is also hampered by the lack of enabling technology, which would achieve a precise control and integration of temperature and meditation/relaxation methods simultaneously, while having the potential to be used in real-life settings at home. Current tech-based solutions to improve sleep either focus on the digital distribution of meditation/relaxation content or on the improvement of quantitative measures of sleep quality. Yet, app-based solutions for sleep are not scientifically validated nor personalized to the client. Solutions based on modern sensing technology (e.g. activity trackers or EEG) are able to more objectively measure sleep quality, but do not provide active solutions to improve it or accelerate sleep onset. Sleep quality and efficiency is largely affected by people’s daily routine and individual cognitive states, such as stress or anxiety levels. Despite progresses in wearable sensing that makes it possible to detect the mentioned individual states with a certain degree of accuracy, and even predict how these states will affect sleep efficiency, no integrated solution links these methods to direct, automated, and personalized interventions for sleep.

### SUMMARY OF INVENTION

The present invention aims at solving or at least reducing the above-mentioned drawbacks of the prior art solutions. To this purpose, it is disclosed a method and system to accelerate sleep onset and/or to improve sleep quality in a subject.

In particular, a first purpose of the system is to precisely and even dynamically optimize the thermal regulation of subject’s body parts in order to facilitate the process of falling asleep and the quality of the sleep. A further purpose of the system is that of synergistically combine meditation-relaxation techniques with thermal regulation of a subject with the aim of enhancing and ameliorating the sleeping time. Still a further purpose of the system is that of supporting the mental and physical relaxation process of a subject. All those aims have been accomplished with the present invention, as described herein and in the appended claims. It is disclosed a method to combine two important components of sleep health, 1) thermoregulation and 2) guided meditation-relaxation practices, into a realistic immersive experience to accelerate sleep onset and improve sleep quality.

In an embodiment, thermoregulation is controlled through the multimodal haptic device described in U.S. Pat. 9,703,381 B2, enclosed herein in its entirety by reference. To improve sleep, in this non-limiting embodiment, the multimodal haptic technology is embedded on a feet device. This is due to the physiological importance of foot temperature in thermoregulation, relaxation, and sleep. Three main parts are disclosed: 1) a procedure to personalize the thermal stimulation needed to accelerate sleep onset in a subject; 2) the merging of thermal stimulation and guided relaxation-meditation techniques into an immersive experience to accelerate sleep onset and enhance sleep quality; and 3) the integration of wearable sensing to develop a recommendation system that can suggest different session durations, according to data recorded from a subject on a given day (closed-loop solution).

In view of the above, according to the present invention a system to accelerate sleep onset and/or to improve sleep quality in a subject is claimed in claim 1, as well as its use for inducing a relaxation state together with thermal regulation in a subject to accelerate sleep onset and/or to improve sleep quality. A non-transitory computer readable medium containing a set of instructions that, when executed by a data processing apparatus comprised in the system cause the data processing apparatus to operate the system to accelerate sleep onset and/or to improve sleep quality in a subject is also disclosed.

Further embodiments of the present invention are defined by the appended claims. The above and other objects, features and advantages of the herein presented subject-matter will become more apparent from a study of the following description.

The method according as disclosed not include surgical or medical steps and the system implementing the method does not require any invasive interaction with the human body. For instance, when reference is made to a “step of monitoring over time the temperature and/or the degree of dilation of blood vessels in the skin of a distal portion of the subject, or variations thereof”, it is meant that such monitoring is made by contacting an external surface of the skin with a device according to the present invention, therefore without practising surgical intervention on the skin in order to reach body vessels with the device.

Moreover, the method as disclosed is not a method and a system for treatment by therapy, meaning that it does not start from a pathological state but from a normal, healthy state. Indeed, the condition of reduced sleep induced for instance by normal circumstances of stress or fatigues do not overlap with symptoms of injury. Furthermore, the system and the method may be applied to induce relaxation and wellness, not only improve sleep.

### DETAILED DESCRIPTION OF THE INVENTION

The subject-matter described in the following will be clarified by means of a description of the several aspects of the invention. It is however to be understood that the scope of protection of the invention is not limited to the described aspects only; to the contrary, the scope of protection of the invention is defined by the claims. Moreover, it is to be understood that the specific conditions or parameters described and/or shown in the following are not limiting of the scope of protection of the invention, and that the terminology used herein is for the purpose of describing particular aspects by way of example only and is not intended to be limiting.

Unless otherwise defined, technical and scientific terms used herein have the same meaning as commonly understood by one of ordinary skill in the art to which this invention belongs. Further, unless otherwise required by the context, singular terms shall include pluralities and plural terms shall include the singular. The methods and techniques of the present disclosure are generally performed according to conventional methods well known in the art and as described in various general and more specific references that are cited and discussed throughout the present specification unless otherwise indicated.

The following description will be better understood by means of the following definitions. As used in the following and in the appended claims, the singular forms “a”, “an” and “the” include plural referents unless the context clearly dictates otherwise. Also, the use of “or” means “and/or” unless stated otherwise. Similarly, “comprise”, “comprises”, “comprising”, “include”, “includes” and “including” are interchangeable and not intended to be limiting. It is to be further understood that where for the description of various embodiments use is made of the term “comprising”, those skilled in the art will understand that in some specific instances, an embodiment can be alternatively described using language “consisting essentially of” or “consisting of.”

In the frame of the present disclosure, the expression “operatively connected” and similar reflects a functional relationship between the several components of the device or a system among them, that is, the term means that the components are correlated in a way to perform a designated function. The “designated function” can change depending on the different components involved in the connection. Likewise, any two components capable of being associated can also be viewed as being “operably couplable,” to each other to achieve the desired functionality. Specific examples of operably couplable include but are not limited to physically interacting components and/or wirelessly interactable and/or wirelessly interacting components and/or logically interacting and/or logically interactable components. A person skilled in the art would easily understand and figure out what are the designated functions of each and every component of the device or the system of the invention, as well as their correlations, on the basis of the present disclosure.

A “haptic device” is any device that exploits haptic technology. As used in the present disclosure, “haptic technology” or “haptics” is a feedback technology which recreates or stimulates the sense of touch by applying forces, pressures, temperatures, electrostimulations, vibrations and/or motions to the user. This mechanical/thermal stimulation can be used e.g. to assist in the creation of virtual objects in a computer simulation, to control such virtual objects, and to enhance the remote control of machines and devices (telerobotics). Haptic devices may incorporate sensors that measure forces, pressures, vibrations, temperature or movements exerted by the user on the interface and vice versa.

As used herein, a “distal body part” refers to body parts in a person including at least one of a hand, a foot, an ankle, a wrist, head and neck. To the contrary, a “proximal body part” refers to the trunk of a subject. Accordingly, “distal body temperature” refers herein to the temperature, or average temperature, of at least on distal body part in a subject including at least one of a hand, a foot, an ankle, a wrist, head and neck. A “proximal body temperature” refers herein to body temperature related to proximal (trunk) body parts such as infraclavicular, thigh, and/or stomach temperature.

For “distal proximal gradient” is herein meant the thermal difference between the body temperature of distal body part/s (or an average thereof) and the body temperature of proximal body part/s (or an average thereof). The distal-proximal temperature gradient (DPG) provides an indirect measure of blood flow in distal skin regions (efficiently regulated by arteriovenous anastomoses), and thereby an indirect index of distal heat loss.

“EEG” is an electrophysiological monitoring method to record electrical activity of the brain. It is typically non-invasive, with the electrodes placed along the scalp, although invasive electrodes are sometimes used, as in electrocorticography. “Thermoregulation” is the ability of an organism to keep its body temperature within certain boundaries, even when the surrounding temperature is very different. A thermoconforming organism, by contrast, simply adopts the surrounding temperature as its own body temperature, thus avoiding the need for internal thermoregulation.

“Polysomnography”, also called a sleep study, is a test used to diagnose sleep disorders. Polysomnography records your brain waves, the oxygen level in your blood, heart rate and breathing, as well as eye and leg movements during the study.

“Sleep onset” is the time passed from lights off to sleep stage N1 and sleep stage N2. This is the lightest stage of sleep and starts when more than 50% of the alpha brain waves are replaced with low-amplitude mixed-frequency (LAMF) activity. Muscle tone is present in the skeletal muscles and breathing tends to occur at a regular rate. This stage tends to last typically between 1 and 5 minutes, consisting of around 5% of the total sleep cycle. Stage N2 represents a deeper sleep phase, when heart rate and body temperate drop. It is characterized by the presence of sleep spindles, K-complexes, or both. The sleep spindles activate the superior temporal gyri, anterior cingulate, insular cortices, and the thalamus. The K-complexes show a transition into a deeper sleep. They are single, long delta waves only lasting for one second. As deeper sleep ensues and the individual moves into N3 stage, all of their waves will be replaced with delta waves. Stage 2 lasts around 25 minutes in the initial cycle and lengthens with each successive cycle, eventually consisting of about 50% of total sleeping time.

“Multimodal” refers herein to the characteristic way by which a haptic device according to the present disclosure provides a user with a feedback. In particular, a multimodal feedback allows a user to experience multiple modes of interfacing with the haptic device. Multimodal interaction is the interaction with a virtual and/or a physical environment through natural modes of communication. This interaction enables a freer and natural communication, interfacing users with automated systems in both input and output. However, in the frame of the present disclosure, the term multimodal refers more specifically to the several modes by which the haptic device can provide tactile feedbacks to a user. The human sense of touch can be divided into two separate channels. Kinaesthetic perception refers to the sensations of positions, velocities, forces and constraints that arise from the muscles and tendons. Force-feedback devices appeal to the kinaesthetic senses by presenting computer-controlled forces to create the illusion of contact with a rigid surface. The cutaneous class of sensations arise through direct contact with the skin surface. Cutaneous stimulation can be further separated into the sensations of pressure, stretch, vibration, and temperature. Tactile devices generally appeal to the cutaneous senses by skin indentation, vibration, stretch and/or electrical stimulation. The device is construed and assembled in order to provide a tactile feedback involving one or more, possibly combined, among kinaesthetic or cutaneous sensations.

The haptic device may comprise one or more sensors for detecting and possibly storing at least a user’s physiological parameter, an environmental parameter or a combination thereof, and is operatively connected with at least one element of the haptic device. A “sensor” as used herein is a device that detects (and possibly responds to) signals, stimuli or changes in quantitative and/or qualitative features of a given system, or the environment in general, and provides a corresponding output. The output is generally a signal that is converted to human-readable display at the sensor location or transmitted electronically over a network for reading or further processing. The specific input could be for instance light, heat, motion, moisture, pressure, or any one of a great number of other environmental phenomena. According to the invention, a sensor preferably comprises a means for detecting and possibly storing user’s physiological parameter, an environmental parameter or a combination thereof. The sensor can therefore comprise a data storage device to hold information, process information, or both. Common used data storage devices include memory cards, disk drives, ROM cartridges, volatile and non-volatile RAMs, optical discs, hard disk drives, flash memories and the like. The information detected and collected by sensors can relate to a user’s physiological parameter such as for instance muscle contraction (including postural muscle contraction), heart work rate, skin conductance (also called galvanic skin response GSR), respiratory rate, respiratory volume, body temperature, blood pressure, blood level of organic/inorganic compounds (e.g. glucose, electrolytes, amino acids, proteins, lipids etc.), electroencephalogram, sweating and so forth. Alternatively or additionally, the information detected and collected by the sensor can relate to environmental parameters such as temperature, humidity, light, sounds and the like.

Preferably, sensors further comprise means for transmitting the detected and possibly stored data concerning the above-mentioned parameters to a computer, and more preferably through a wireless connection. “Wireless” refers herein to the transfer of information signals between two or more devices that are not connected by an electrical conductor, that is, without using wires. Some common means of wirelessly transferring signals includes, without limitations, WiFi, bluetooth, magnetic, radio, telemetric, infrared, optical, ultrasonic connection and the like.

In one embodiment, sensors further comprise means for wirelessly receiving a feedback input from a computer able to regulate the functioning of the device. In one embodiment, sensors are operatively connected to a display unit and/or to a manifold. The main actuation unit controls the cells in the device without any cable (depending on the configuration, but at least in a configuration where the valves are on a main manifold). The main actuation unit could feature a printed circuit board (PCB) with e.g. a microcontroller controlling all the components (i.e. pumps, valves, sensors and any other component mounted on a main manifold). For this reason, the board manages the low-level functions such as a closed feedback loop controlling the pressure and possibly temperature in the cells. The board can be seen as a driver for the device communicating wirelessly with a computer or a mobile phone managing high-level functions.

A “closed-loop system”, also known as a feedback control system, refers herein to a control system which uses the concept of an open loop system (in which the output has no influence or effect on the control action of the input signal) as its forward path but has one or more feedback loops (hence its name) or paths between its output and its input. The reference to “feedback” means that some portion of the output is returned back to the input to form part of the system’s excitation. Closed-loop systems are usually designed to automatically achieve and maintain the desired output condition by comparing it with the actual condition. It does this by generating an “error” signal which is the difference between the output and the reference input. In other words, a closed-loop system is a fully automatic control system in which its control action is dependent on the output in some way.

The expression “haptic profile” is herein used to intend the sequence of instructions necessary to functionally operate a haptic device according to one or more input data. More precisely, a “haptic profile” refers herein to the instructions encoding an activation, such as a spatio-temporal activation, of a plurality of operatively connected tactile displays in a display unit, said activation being coherent with the audio profile of an audio file. A haptic profile encodes the activation pattern of a display unit based on the type of sensory tactile to be provided (i.e. pressure, possibly and in some embodiments preferably combined with temperature), the number and identity of displays and their relative positioning, the duration of stimulation of the displays, the synchrony/asynchrony of a stimulus onset among the displays and the percentage of activation overlap between the displays in a unit; at the same time, the modulation of those several parameters is tightly linked to the output of an audio processing (referred to herein as “audio profile”), which in turn gives rise to a haptic profile via an audio-to-haptic, or video-to-haptic conversion, in which an elaboration of processed frequencies and/or amplitudes of an audio signal produces an audio profile and therefore a precise signature of tactile display activation.

For “computer-readable data carrier” or “computer-readable medium” is herein meant any available medium that can be accessed by a processor and may include both a volatile and non-volatile medium, a removable and non-removable medium, a communication medium, and a storage medium. A communication medium may include computer readable instructions, data structures, program modules or other data in a modulated data signal such as a carrier wave or other transport mechanism, and may include any other form of an information delivery medium known in the art. A storage medium may include RAM, flash memory, ROM, erasable programmable read-only memory (“EPROM”), electrically erasable programmable read-only memory (“EEPROM”), registers, hard disk, a removable disk, a compact disk read-only memory (“CD-ROM”), or any other form of a storage medium known in the art.

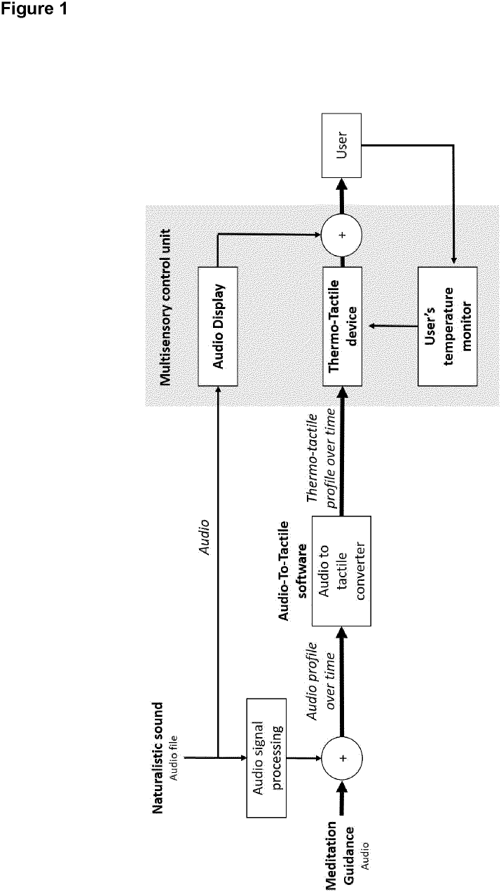

In one aspect, the present disclosure features a method to accelerate sleep onset and/or to improve sleep quality in a subject, the method comprising the steps of (FIG. 1):
a) reproducing an audio file, with or without a video file, via an audio/video device; and
b) providing thermo-tactile stimuli to a distal portion of the subject to increase the degree of dilation of blood vessels in the skin of said distal portion, wherein the thermo-tactile stimuli are provided through a haptic device according to a haptic profile obtained by said audio file, thereby inducing a relaxation state together with thermal regulation in said subject to accelerate sleep onset and/or to improve sleep quality. The distal portion of a subject, according to the disclosure, comprises at least one of a hand, a foot, an ankle, a wrist, head and neck.

The method of the disclosure is performed on a subject, particularly a human being, that wants to prepare for, is prepared or otherwise ready to start a sleeping phase. In particular, the subject is in a sitting position, or laying down on an appropriate sleeping support, such as a bed or a mat, in a suitable sleeping position depending on the subject’s needs. The method of the disclosure is therefore implemented on a subject that is about to sleep, i.e. in close temporal proximity to the entry of the subject into an N1 sleeping phase, such as between 60 minutes and 1 minutes before an N1 sleeping phase, and/or during any or every sleeping phase of the subject.

The method of the disclosure, accordingly, is construed and configured to prepare, accompany, favour, accelerate and/or ease the transition from wakefulness to sleep, as well as, in certain embodiments, to maintain and/or improve the sleep phases or the quality thereof, including augmenting the length of one or more sleep phases, facilitating the passage from one sleep cycle to another, smoothing the process of falling asleep again and the like.

The method of the disclosure is preferably implemented via an integrated system, which represents another aspect of the disclosure, said system comprising:
a) a haptic device comprising a plurality of tactile displays configured to provide thermo-tactile stimuli to a user;
b) at least one audio/video device configured to reproduce an audio file, with or without a video file; and
c) a data processing apparatus operatively connected with said haptic device and said audio/video device, the data processing apparatus having a processor comprising instructions configured to operate the system to perform the method of the disclosure. The data processing apparatus of the can comprise any suitable device such as computers, smartphones, tablets, voice-activated devices (i.e. smart speakers/voice assistants) and the like. In preferred embodiments, the system further comprises sensors operatively connected with the data processing apparatus, the sensors being configured to measure the temperature of at least a distal portion of the subject, such as at least one of a hand, a foot, an ankle, a wrist, head and neck, or variations thereof.

In some embodiments, the at least one audio/video device configured to reproduce an audio file comprises one or more speakers. The computer’s processor may transmit an audio signal to speakers, which in turn outputs audio effects. Alternatively, earphones or headphones could be used.

A suitable haptic device according to the present disclosure is flexible and adaptable to the user’s anatomy, and can provide thermo-tactile haptic feedbacks. Generally, the device comprises at least an actuation unit connected to a flexible display unit. The actuation unit pneumatically and/or hydraulically controls the pressure and temperature of a fluid medium, such as liquid or a gas, to provide tactile and temperature cues to the subject touching the display. The tactile cues are created by controlling the shape of a flexible membrane of the display through the fluid medium pressure. In one embodiment, contrary to known tactile displays that make use of several rigid actuators in order to obtain a multimodal feedback, a haptic device features one single actuation system that generates the integrity of the multiple haptic feedbacks, both tactile and proprioceptive (e.g. thermal cues).

In one embodiment, thermal cues are provided by the heat exchange between the fluid medium and the user’s skin through the same membrane. The temperature of the fluid medium stream flowing in the display is achieved by mixing several fluid streams at specific temperatures. These fluids are heated or cooled to specific temperatures using e.g. Peltier elements, and can be stored in buffer tanks. The medium flow and pressure are controlled using a combination of micro pumps and valves. The flexible display is composed of an array of cells named tactile displays or cells. The number of cells and their disposition is modular in order to adapt the cell density to the type and surface of skin. In one embodiment, the medium pressure and temperature can be controlled in each individual display cell using a system of valves and manifolds, possibly embeddable in a compact actuation unit. The tactile display can have different functional shapes adapted to the user’s anatomy.

In one embodiment, the haptic device is the one described in U.S. Pat. 9,703,381 B2, which is particularly suitable for the target applications of the system and method of the disclosure.

According to a main embodiment of the disclosure, thermo-tactile stimuli provided to a distal portion of the subject, in order to increase the degree of dilation of blood vessels in the skin of said distal portion, are furnished to the subject by activating or otherwise operating a haptic device in contact with the distal portion of the subject while providing heat. The heat exchange between the haptic device and the subject’s skin is preferably performed by tactile displays or cells, and the amount of heat provided by the haptic device can be variable and dynamically regulated in order to keep a constant or variable degree of blood vessel dilation. Typically, a haptic device suitable for performing the method of the disclosure is able to heat the subject’s skin of up to 40-45° C. to induce an appropriate blood vessel dilation for accelerating sleep onset and/or improving sleep quality in said subject.

In one embodiment, the method of the disclosure further comprises a step of monitoring over time the temperature and/or the degree of dilation of blood vessels in the skin of a distal portion of the subject, or variations thereof. In this embodiment, a system according to the disclosure comprises temperature and/or blood pressure sensors configured to measure, preferably in real-time, the temperature and/or the degree of dilation of blood vessels, as well as the blood flow of the subject’s body part skin, and in contact with said subject’s skin undergoing the method of the disclosure, said sensors operating in a closed-loop with a data processing apparatus to dynamically set the heat exchange between the device and the subject in order to maintain an appropriate blood vessel dilation.

In preferred embodiments of the disclosure, the reproduced audio file comprises at least an audio track encoding a voice and an audio track encoding a sound.

In preferred embodiments of the disclosure, the haptic profile is obtained by:
a) processing an audio signal derived from an audio file, thereby obtaining at least one profile of frequencies and/or amplitudes of said audio signal; and
b) converting said frequencies and/or amplitudes profiles into a haptic profile.

In some embodiments, the processed audio signal is obtained from an audio file encoding a sound.

In some embodiments of the disclosure, the method comprises a step of measuring and/or monitoring the temperature of at least a portion of the trunk of the subject, and the temperature of a distal portion of the subject.

In some embodiments of the disclosure, the method foresees providing thermo-tactile stimuli, including thermal and pressure gradients, on the subject skin in a spatio-temporal fashion.

According to a particular embodiment of the disclosure, the thermo-tactile stimuli have a duration based on physiological, movement and/or psychological parameters of the subject. Said parameters can be obtained in real-time with the use of wearable sensors, off-line (by uploading files concerning said parameters into a data processing apparatus according to the disclosure, as described hereinafter), or combinations thereof. This embodiment is construed to attain a so-called “activity-dependent personalization” of the method of the disclosure. As sleep research has shown that individual state’s during the day, such as stress or anxiety, plays a significant role in sleep quality, and in parallel computational sleep research has shown that sleep quality and efficiency can be predicted by activity data and other bodily signals such as heart rare, breathing patterns and body temperature, the method of the disclosure foresees a “recommendation system” that suggest the ideal duration of the sleep-inducing session, selected according to a participant’s wearable day-by-day sensing data profile. This allows to improve sleep quality across different days. For instance, in order to maintain similar sleep onset and quality during different nights, sleep-inducing sessions may be programmed to last longer after a stressful day. It is anticipated that different duration of the sleep-inducing condition will be needed to achieve a similar sleep improvement in different days. For examples, it is anticipated that longer duration of the sleep-inducing session will be needed after days of intense stress. Differing from passive sensing technologies, the disclosure allows to link individual perception and individual mental and physiological states to a direct intervention to accelerate sleep onset and improve sleep quality.

Still another aspect of the disclosure relates to a non-transitory computer readable medium containing a set of instructions that, when executed by data processing apparatus of the system of the invention, cause said data processing apparatus to operate the system to perform a method according to the disclosure. Further, one aspect of the invention relates to a data processing apparatus comprising the non-transitory computer readable medium of the disclosureinvention.

In embodiments, the instructions contained by the non-transitory computer readable medium comprise:
i) receiving and/or processing data regarding the temperature of at least one distal portion of the subject, or variations thereof;
ii) operating an audio/video device to reproduce an audio file, with or without a video file;
iii) receiving and/or processing data regarding a haptic profile obtained by said audio file; and
iv) operating a haptic device to provide thermo-tactile stimuli to at least one distal portion of the subject.

In some embodiments, the instructions contained by the non-transitory computer readable medium further comprise:

v) receiving and/or processing data regarding the temperature of at least a portion of the trunk of the subject or variations thereof, measured by sensors placed on said subject, and/or receiving and/or processing data regarding a distal-proximal temperature gradient of the subject, defined as the difference between the temperature of at least a portion of the trunk of the subject and the temperature of at least one distal portion of the subject.

In one embodiment, the apparatus comprises memory storing software modules that provide functionality when executed by the processor. The modules include an operating system that provides operating system functionality for the apparatus. The modules further include a haptic conversion module that converts an audio signal into a haptic profile which encodes information as to how to operate the haptic device, as disclosed in more detail below. The apparatus, in embodiments that transmit and/or receive data from remote sources, further includes a communication device, such as a network interface card, to provide mobile wireless communication, such as Bluetooth, infrared, radio, Wi-Fi, cellular network, or other next-generation wireless-data network communication. In other embodiments, communication device provides a wired network connection, such as an Ethernet connection or a modem.

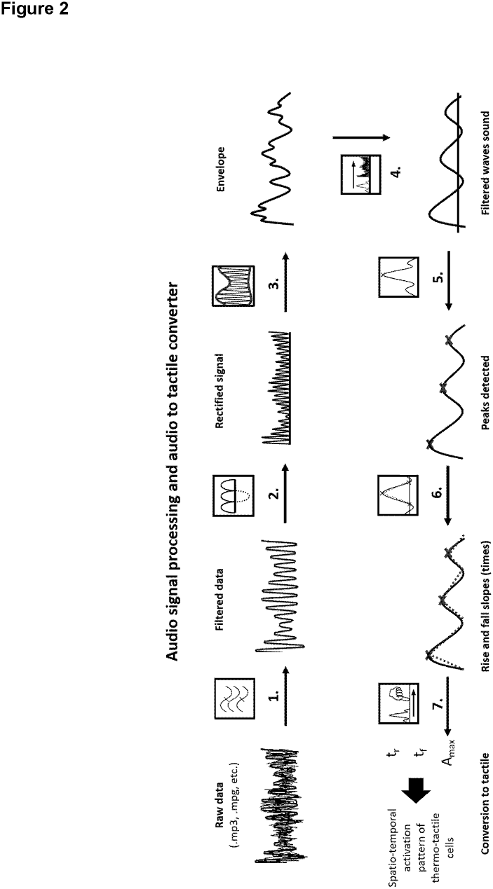

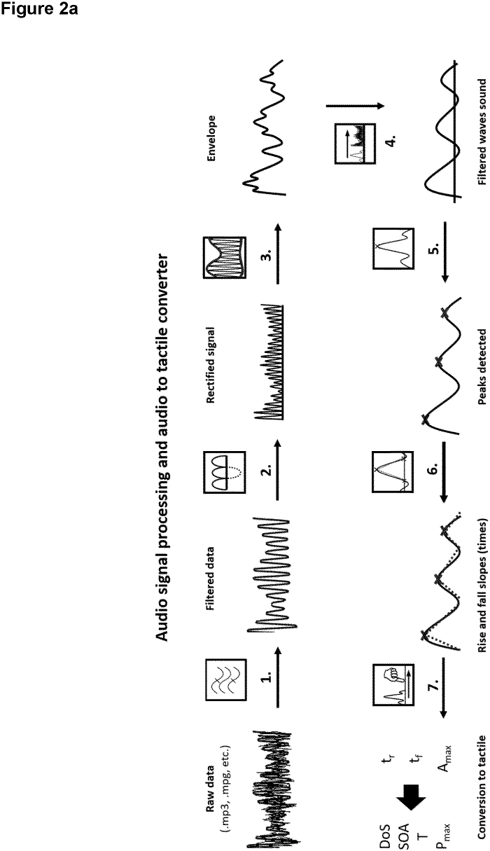

According to this embodiment, the data processing apparatus executes a first step of the method by processing an audio signal derived from an audio file so to obtain at least one profile of frequencies and/or amplitudes of said audio signal (FIG. 2). According to one embodiment, an envelope of the audio signal is first extracted. An envelope can be extracted using all frequencies of an original audio signal or a filtered version of the original audio signal. However, the envelope itself does not have the same frequency content as the original audio signal.

In embodiments of the disclosure, to obtain a profile of frequencies and/or amplitudes of said audio signal the processing comprises applying a bandpass filter on the input audio file, broadly centred around a desired frequency. The signal is then rectified, and a low-pass filter is applied to obtain the envelope of the signal. In some embodiments, the envelope is then down-sampled, and a noise threshold amplitude is applied. In some embodiments, a window function such as a convolved Hanning window, which time constant is based on prior knowledge on the desired signal, is then applied.

Once obtained a filtered envelope, the computer apparatus executes a second step of the method by converting the frequencies and/or amplitudes profile in the form of a filtered envelope into a haptic profile by a haptic converter module. As the amplitude of the envelope encodes the “strength” of an audio signal, which will be later on converted into the “strength” of a haptic feedback, the peaks and the valleys of the envelope (i.e. its local maxima and minima) are tracked. Prior knowledge on the type of the desired haptic feedback can also be used to fine tune the peak research by defining minimum times between peaks and minimum peak amplitudes.

Once the peaks and valleys located, the computer apparatus executes a further step of the method by operating the haptic device according to the obtained haptic profile. By “operating the haptic device according to the obtained haptic profile” is herein meant that the processor transmits a signal associated with the obtained haptic profile to the haptic device, which in turn outputs haptic sensations to a subject.

As a way of example, a sound of waves can be converted into a tactile stimulation provided with a thermo-tactile display composed of e.g. 3 cells (each cell is controllable in pressure and temperature).

Once the peaks located, it is possible to estimate the onset of the wave, i.e. the last occurrence before the peak when the amplitude of the envelope goes over the noise threshold, and the end of the wave, i.e. the first occurrence after the peak when the amplitude of the envelope falls under the noise threshold. From there, the rise and fall time of the waves are also derived and the wave is fully characterized. In order to convert the waves into a tactile stimulation spread over three cells, three waves’ parameters are considered: the rise time (tᵣ), the fall time (t_{f}), and maximum amplitude of the wave (Aₘₐₓ).

To give the sensation that the wave is moving on the user’s skin, the rise and fall time of the wave are encoded as a spatio-temporal pattern of activation of the 3 display cells, thus resulting in two distinct tactile patterns for the rise and fall of the wave. To ensure that the user perceives a fluent tactile movement encoding a wave, and not three discrete stimulations, the activation of the cells overlap in time. The maximum amplitude of the wave is normalized by a maximum tactile pressure value (determined by the user or the maximum pressure available in the display) and used as input pressure command for the cells.

However, in another set of embodiments, the haptic profile has been previously determined or retrieved in a database, and can be directly used to operate the haptic device.

In some embodiments, the instructions processed by the non-transitory computer readable medium further comprise:

vi) receiving and/or processing data regarding physiological, movement and/or psychological parameters of the subject.

As will be apparent for a person skilled in the art, based on what described, still another aspect of the disclosure relates to the use of a system according to the invention for inducing a relaxation state together with thermal regulation in a subject to accelerate sleep onset and/or to improve sleep quality.

In a more general aspect:

The haptic device may be flexible, for adaptation to the distal portion, and may include a plurality of cells separately controlled in pressure and/or temperature and/or activation time and/or duration time, preferably by means of pressure and/or temperature of a medium in the cells. The plurality of cells is arranged on the distal portion to define a cell pattern.

The method to accelerate sleep onset and/or to improve sleep quality may include the steps of:
-determining a wave profile of frequencies and/or amplitudes of the audio signal, including peaks and valleys,
-identifying a plurality of wave parameters, at least including a rise time, i.e. the time when the wave start to rise, a rise duration, i.e. how long the wave rises, a fall time, i.e. the time when the wave start to fall, a fall duration, i.e. how long the wave falls, a slope of the wave and an amplitude of the wave, and transforming the plurality of wave parameters into haptic profile parameters for driving the plurality of cells.

For instance, based on the rise time, the rise duration, the slope at rise time and the amplitude of the wave taken as wave parameters, the haptic profile parameters are determined as follows.

The haptic profile parameters for a first cell in the cell pattern include the activation start time of the first cell, corresponding to the rise time of the wave, the activation duration of the first cell, corresponding to a part (or percentage) of the rise duration of the wave.

For instance, the activation duration of a cell i in the pattern of n cells, which is the time the cell i remains inflated with the medium, is from its activation time during the rise of the wave to its deactivation time during the fall of the wave. The activation duration di of each cell i can be derived from its activation time tai and its deactivation time tdi as follows.

The activation time tai of each cell i is:

$$t a i = t r + \left(d r / m\right) * i ,$$

where m<n, dr is the rise duration of the wave, and tr is the rise time of the wave. The deactivation time tdi during the fall of the wave of each cell i is:

$$t d i = t f + \left(d f / m\right) * i ,$$

where m<n, df is the fall duration, and tf is the fall time of the wave.

The activation duration of each cell is

$$d i = t d i - t a i .$$

The activation duration of the first cell may correspond to the activation duration of all the other cells in the cell pattern. However, the activation time of one cell in the pattern is preferably different from the other cell in the pattern, for instance based on a delay with respect to a previous cell in the pattern. The delay may be higher depending on the position of the cell in the pattern; for instance, the delay of activation of the 3^{rd} cell with respect to the 1^{st} cell in the pattern may be greater than the delay of activation of the 2^{nd} cell with respect to the 1^{st} cell in the pattern.

Further than activation time and actuation duration the haptic profile parameters include the pressure and/or temperature values to set in the cells. Still with reference to the example above, taking the part of the wave (wave pattern) from a valley to a peak, and starting from rise time, the pressure and/or the temperature of the medium in the cell may be determined as a function of the slope and/or the amplitude of the wave pattern. Accordingly, due to the delays, the pressure and/or temperature of the cells along the cell patterns (and therefore along the distal part of the body) may be different.

All the above-mentioned haptic profile parameters, which have been given as an example for the present disclosure, are used to set signals for driving the cells. Accordingly, the step of providing thermo-tactile stimuli includes transmitting a plurality of signals to the plurality of cells, each plurality of signals being associated to a plurality of haptic profile parameters and being directed to one of the plurality of cells in order to drive it as to its actuation time, duration, pressure and/or temperature, etc.

In the example given above, the wave pattern starting at rise time is transformed into haptic profile parameters adapted to provide a spatio-temporal activation pattern of the plurality of cells. In a same way, the wave pattern starting at fall time may be transformed into haptic profile parameters adapted to provide a spatio-temporal activation pattern of the plurality of cells.

The transmission of signals to a first cell is asynchronous with respect to signals transmitted to another cell. For instance, transmission of signals directed to one cell begins before an end of transmission of other signals to another cell. However, nothing prevents that signal directed to one cell are transmitted at the same time when signals directed to another cell are transmitted, in order to provide overlapping actuations of the cells.

Duration of the thermo-tactile stimuli corresponds to duration of the signals. However, duration of signals directed to one cell may be different or equal to duration of other signals to another cell of the plurality of cells.

Pressure and/or temperature of the thermo-tactile stimuli is set based on body parameters measured in a body portion different from the distal portion.

To summarize, by providing also another example, in order to convert the waves into a tactile stimulation spread over a plurality cells (for instance three cells as depicted in FIGS. 2a and 2b), a pluraity wave’s parameters are considered: the rise time and rise duration, the fall time and fall duration, the maximum amplitude of the wave, its slope, etc. To give the sensation that the wave is moving on the user’s skin, the rise and fall time are encoded as a spatio-temporal pattern of activation of the plurality cells, for example resulting in two distinct tactile patterns for the rise and fall of the wave. To give the impression to the user of a fluent tactile movement encoding a wave, and not of a plurality (three) discrete stimulations, the activation of the cells overlap in time. The overlap is determined by the body part stimulated (because of the different spatial resolutions in different body parts) and by the distance between the display’s cells. For a given rise (and fall) time of the wave (tᵣ or t_{f}), the duration of stimulation of the cells (DoS) and the stimulus-onset asynchrony (SOA) may be determined based on the percentage of overlap (Oₚ) with the following equations:

$${\text{t}}_{\text{r}} = 3 \times \text{DoS} \times {\text{O}}_{\text{p}} \qquad \text{(1)}$$

$$\text{SOA =} \left(1 {\text{- O}}_{\text{p}}\right) \times \text{DoS} \qquad \text{(2)}$$

Preferably, the maximum amplitude of the wave is normalized by a maximum tactile pressure value (determined by the user or the maximum pressure available in the display) and used as input pressure command for the cells. With reference to this possible example, reference to FIGS. 2a and 2b is also made.

The skilled person may understand that features disclosed with reference to the more general aspect mentioned above may be applied in any one of the embodiments of the method and system of the present disclosure, and therefore these features are not repeated for all the embodiments

### EXAMPLES

To describe and illustrate more clearly the present disclosure, the following examples are provided in detail, which however are not intended to be limiting of the scope of protection as defined in the claims.

### Thermal Personalization

Previous studies have shown that the degree of dilation of blood vessels in the skin of the hands and feet, which increases heat loss at these extremities, is the best physiological predictor for the rapid onset of sleep (Kräuchi et al., 1999). As a proxy to monitor this parameter, these authors calculated the distal-proximal temperature gradient (DPG), a measure of blood flow in distal skin regions (efficiently regulated by arteriovenous anastomoses) that provides an indirect index of distal heat loss. To this aim, temperature sensors are preferably integrated within a multimodal haptic system. Based on this, the exact thermal stimulation pattern for each participant is selected, before each section employing thermal stimulation, through a Bayesian optimization approach aiming at the personalized maximization of the distal vasodilation (i.e. maximization of the DPG).

### Multisensory and Cognitive Stimulation to Enhance Sleep

After the thermal personalization phase, the user goes through an immersive experience phase. The user sits, for example on a chair few minutes before going to bed, or lays down, for example on a bed, place his/her feet and/or hands on the multimodal haptic device, put on his/her headphones, and close his/her eyes. As a way of example, water waves of an ocean or lake, according to the user preferences, are simulated, through touch, temperature, and sound, under the user’s feet and/or palms. At the same time, a guiding voice helps the user follow a meditation or relaxation practice.

The described multisensory scenario (water waves simulated through both touch and sound) can increase the immersion of the meditation (e.g., the sensations of being located on a lake shore and being touched by water waves) and improve the meditation experience (e.g. increased focus and absorption into the meditation).

It is anticipated that in an experimental study (40 naïve healthy participants - 20 males; all women will be tested at the same time relative to their menstrual cycle - with normal and regular sleep-wake habits) the main experimental condition - Sleep-inducing -incorporates personalized application of thermal stimulation under both feet and relaxation-meditation characterized by immersion into a beach soundscape and a pre-recorded guidance prepared by meditation expert. Right before going to bed, participants undergo 10 - 20 minutes of the Sleep-inducing condition or the following three control conditions:
1) Guided relaxation meditation without thermal stimulation (same guided relaxation meditation used during the Sleep-inducing condition, but without thermal stimulation nor soundscape);
2) Thermal stimulation without guided relaxation meditation (same thermal stimulation used in the Sleep-inducing condition but without guided relaxation meditation nor soundscape);
3) Control: participants will stay in the same position prior to sleep for the same amount of time but without any of the other stimulations.

Before the experiment, sleep quality and quantity are assessed by self-rated sleep questionnaires and wrist actimetry over three consecutive nights before each experimental session. A battery of standardized questionnaires to assess anxiety, mood, relaxation, vigilance, etc., is filled out by each participant before inclusion in the experiment, and re-assessed before and after each night.

EEG data and/or sleep polysomnography can be scored over 30s epochs, according to standard criteria (e.g. AASM Manual for the Scoring of Sleep and Associated Events: Rules, Terminology and Technical Specifications (2020), url:https://aasm.org/clinical-resources/scoring-manual/), by two experienced scorers blind to the experimental conditions. Several sleep parameters may be calculated: latencies to stage N1 (from lights off), and N2 (from first N1 period), time and percentage of each sleep stage, total sleep time (TST; sum of the time spent in different sleep stages), total sleep period (SPT; total time from sleep onset to final awakening, including intra-sleep wake intervals). Sleep efficiency is defined as TST/SPT x 100. Sleep spindles will be visually quantified at Cz contact referenced against mastoid channels, based on their typical fusiform morphology. Planned comparisons between the Sleep-inducing condition and the control conditions will be performed using paired 2-tailed t-tests, based on the following specific hypotheses.

It is anticipated that the Sleep-inducing condition would accelerate sleep onset (i.e. shorter duration of stage N1 sleep and a reduction of stage N2 latency) as compared to the other experimental conditions. Based on the combination of personalized thermal stimulation with guided relaxation, it is anticipated to achieve at least a 15% accelerated sleep onset when comparing Sleep-inducing vs control 2 and at least 20% when comparing Sleep-inducing vs control conditions 1, 2 and 3). It is also anticipated that the sleep-inducing condition will promote consolidated sleep by increasing the duration of stage N2 and N3 sleep and by enhancing sleep oscillations (spindles and slow waves) as compared to the other control conditions. Finally, it is anticipated better overall sleep quantity and quality (as assessed through questionnaire) in the Sleep-inducing than the other control conditions.

Immersive audio and thermo-tactile stimulation provides a more restoring (less fragmented) nap that increases vigilance (shorter reaction times). 23 naïve healthy participants (15 female; age: 25.7 +/- 5.1) with normal and regular sleep-wake habits took part in this study. Before the experiment, sleep quality and quantity were assessed by self-rated sleep agenda and wrist actimetry over three consecutive nights before each experimental session. A battery of standardized questionnaires to assess the daytime sleepiness, subjective sleep quality, depressive symptoms, and scale of health, was filled out by each participant before inclusion in the experiment. All participants were self-reported naive meditators (they had never practiced meditation or occasionally took one or two meditation session in their lifetime) and non-usual nappers (maximum 1 occasional nap per week). They had to be free of medications known to affect sleep or the circadian system, cardiovascular medication, and psychotropic medication, except for oral contraceptives for females and had no history of neurological or psychiatric disorder.

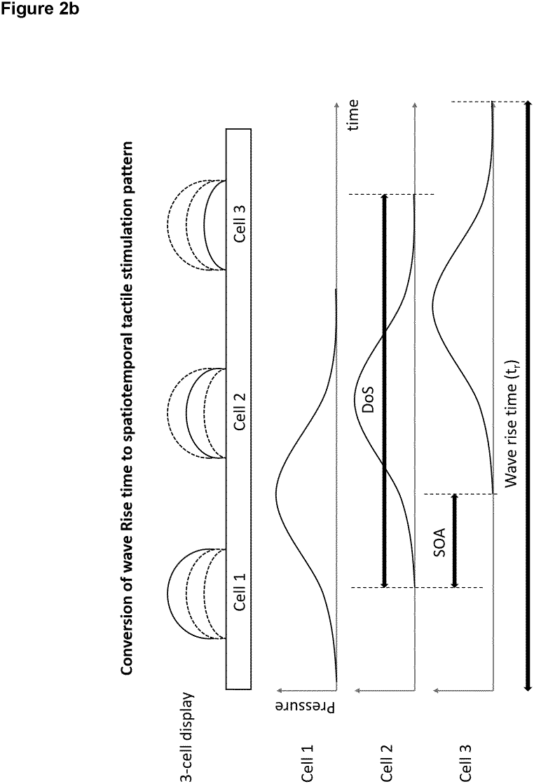

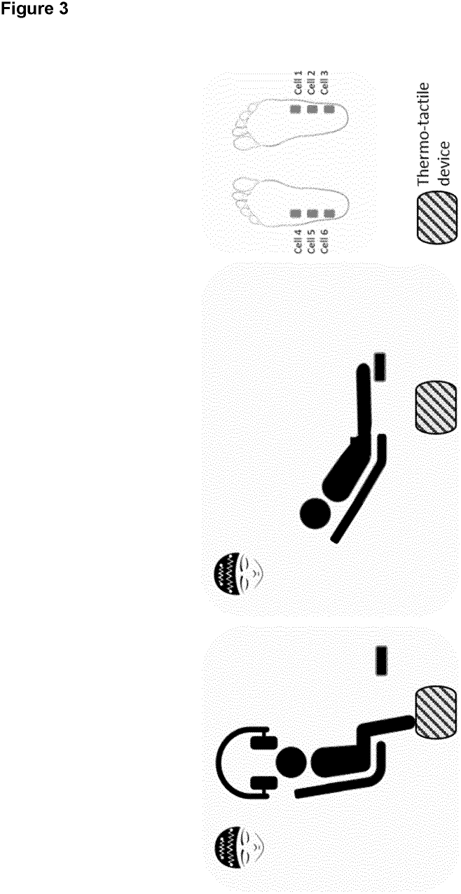

During three different days, each participant completed three different recording sessions, corresponding to three different experimental conditions where Electroencephalogram (EEG) was recorded throughout the session. Each session started with a meditation phase of 12 minutes in dim light: the participant sit on a relaxing chair, remove their shoes while keeping socks on, place their feet on the multimodal haptic device and covered them with a blanket, wore the EEG device as well as headphones, and close his/her eyes (FIG. 3). Each experimental condition lasted the same amount of time. The participant underwent the three following conditions in a pseudo-randomized and counter-balanced order:
1) Thermo-tactile condition: a multisensory immersive meditation scenario - the main experimental condition. The multisensory scenario (water waves simulated through both touch and temperature below the feet and sound) used a both soundscape of a seashore and thermal-tactile stimulation with temperature of 45° C. At the same time, a pre-recorded guidance prepared by meditation expert helped the user to follow a sleep-inducing meditation practice (FIG. 4);
2) Sound meditation: a guided relaxation meditation with soundscapes, without thermal nor tactile stimulation. The meditation and the soundscapes were the exact same used in the Thermo-tactile condition;
3) Rest: participant was asked to focus on his/her breath, while remaining in the same position prior to sleep. No meditation, nor thermal, tactile, or auditory stimulation was administered during this experimental condition.

In order to create the Thermo-tactile condition, the soundscape (i.e. sound of natural water waves) was converted into a tactile stimulation provided under the user’s feet, through a thermo-tactile display composed of 6 cells (each cell is controllable in pressure and temperature), 3 for each foot.

This was done by following the steps described in FIG. 2, where the maximum amplitude of the wave was normalized by a maximum tactile pressure value of 300 mPa, whereas the temperature of the cells was set to 45° C. The frequency of the naturalistic waves was chosen to be 0.2 Hz, as inertial or sound stimulation around this frequency has been shown to facilitate sleep and improve sleep quality (Bayer, L., Constantinescu, I., Perrig, S., Vienne, J., Vidal, P. P., Mühlethaler, M., & Schwartz, S. (2011). Rockingsynchronizes brain waves during a short nap. Current biology, 21(12),R461-R462; Cordi, Maren Jasmin, Sandra Ackermann, and Björn Rasch. “Effects of relaxing music on healthy sleep.” Scientific reports 9.1 (2019): 1-9.

Immediately after the end of the meditation session, the participant opened his/her eye, placed his/her feet on a footrest, reclined the back of the chair and close his/her eyes again for a duration of 45 minutes during which he/she may have felt asleep. Lights were turned off and marked the start of the napping time.

At the end of the 45 minutes, the subject was awakened, lights were turned on, and the participant had 15 minutes to emerge from his/her slumber. The participant ended the session with a computer-based task (Psychomotor Vigilance Task; PVT) to assess vigilance. The PVT is a reaction-time task where the participant is asked to presses a button as soon as a sign appears on the computer screen. Shorter reaction times indicates higher level of vigilance.

EEG data and/or sleep polysomnography were scored over 30s epochs, according to standard criteria (e.g. Iber C, Ancoli-Israel S, Chesson A, Quan S, for the American Academy of Sleep Medicine. The AASM manual for the scoring of sleep and associated events: rules, terminology and technical specifications. 1st ed. American Academy of Sleep Medicine: Westchester; 2007), by two experienced scorers blind to the experimental conditions. Several sleep parameters were calculated: latencies to stage N1, N2, N3 and NREM (from lights off) if the stage was reached, time and percentage of each sleep stage, total sleep time (TST; sum of the time spent in different sleep stages), total sleep period (SPT; total time from sleep onset to final awakening, including intra-sleep wake intervals). Sleep efficiency was defined as TST/SPT x 100. Sleep fragmentation and related parameters were computed based on the sleep hypnogram. For normally distributed data (Kolmogorov-Smirnov test), ANOVA comparison and post-hoc planned comparisons between the Thermo-tactile condition and the control conditions were performed using paired 2-tailed t-tests. Otherwise, non-parametric Wilcoxon test were used.

15 subjects were included in the analysis, the other being removed either due to bad signal quality or issues during the recordings.

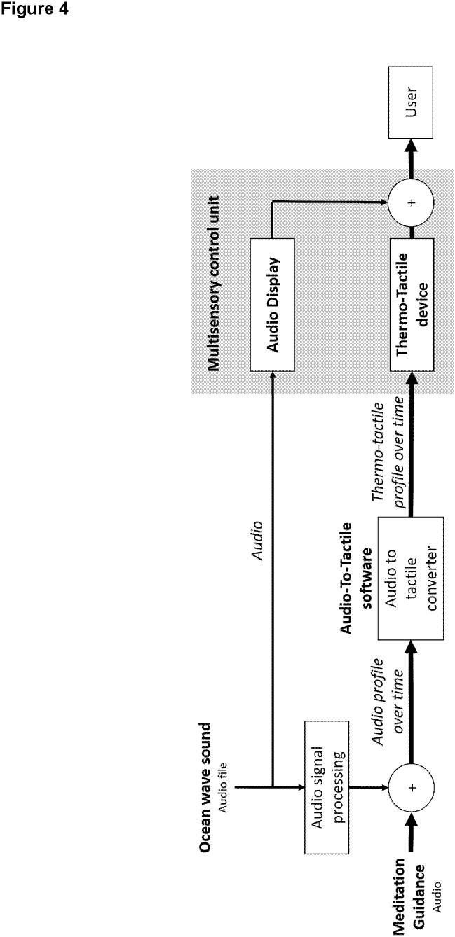

FIG. 5 shows the number of sleep stages transition (fragmentation: the lower the fragmentation the better the sleep quality) during sleep, an indicator of sleep quality (N=15). The repeated measure ANOVA with the three experimental conditions (Rest, Sound, Thermo-tactile) as factors showed a main effect of sleep stage (F(2,28=3.95, p<0.05). Crucially, there was a significant decrease of fragmentation for the Thermo-tactile compared to the Sound meditation condition (two-tailed paired t-test, p<0.05), and a quantitative decrease for Thermo-tactile compared to the Rest condition. Vertical bars denote standard errors of the mean. Thus, Thermo-tactile condition promoted consolidated sleep stages by reducing transitions between sleep stages during the nap. This suggests a better sleep quality during the Thermo-tactile condition as compared to the other conditions.

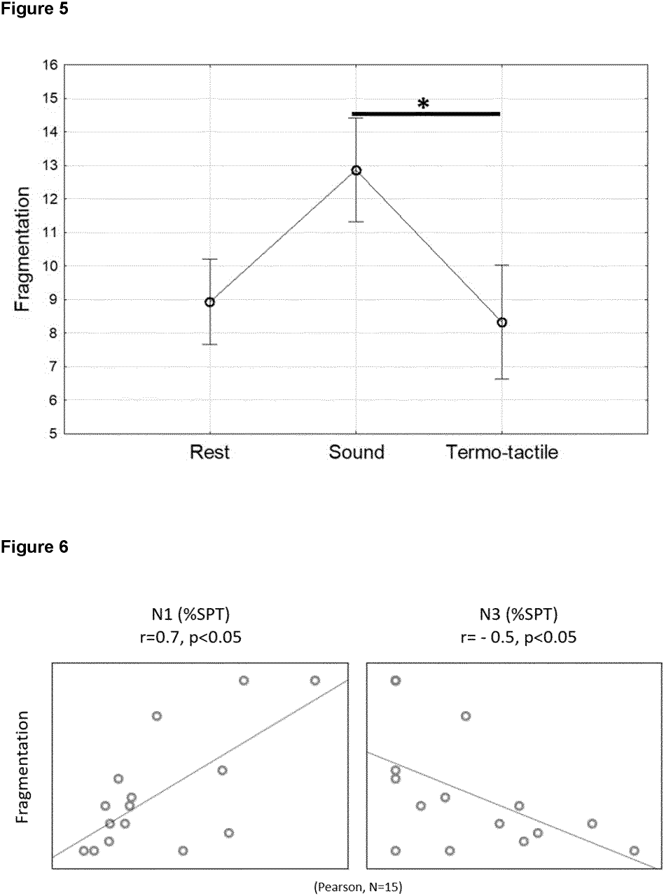

FIG. 6 shows two significant Pearson’s correlation for the Thermo-tactile condition fragmentation, percentage of time spent in N1 over SPT (r = 0.7, p<0.05) and percentage of time spent in N3 over SPT (r = -0.5, p<0.05; N=15). Thus, during the Thermo-tactile condition, the lower the fragmentation the lower the percentage of time spent in light sleep stage (N1) over SPT; and the lower the fragmentation the higher the percentage of time spent in deep sleep, more restoring, stage (N3) over SPT. This strengthens the link between low fragmentation and deeper or more restoring sleep.

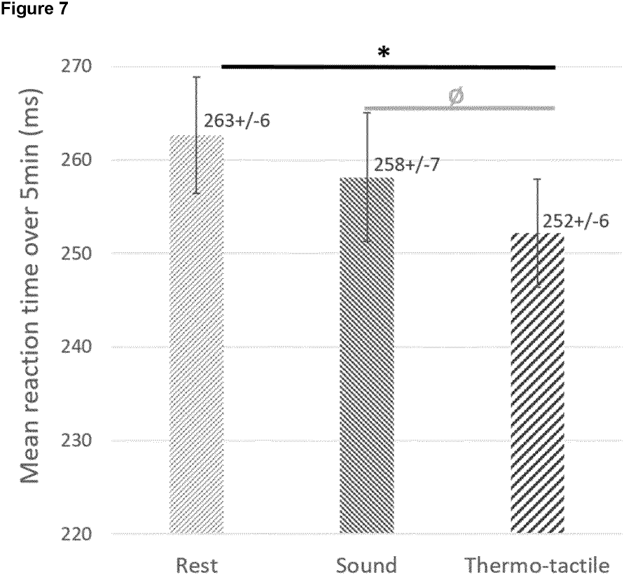

FIG. 7 shows the mean reaction time after the nap, assessed with a computer based task (PVT) (N=12), per experimental condition. Two-tailed paired t-test showed a significant difference in reaction time for Rest vs Thermo-tactile (p<0.02), and a tendency for Thermo-tactile to lower the reaction time compared to the Sound condition. Thus, the Thermo-tactile condition impacted not only neural sleep metrics but also behavioural data. Thermo-tactile condition improves the vigilance after the nap by reducing the participant reaction time to a visual stimulus. This results are in line with the neural results on fragmentation and speak in favour of a more restoring sleep (nap) during the thermo-tactile condition than the other experimental conditions.

In summary, the Thermo-tactile condition improved sleep as assessed by both EEG and behavioural data: it enhanced the propensity to attain and stay in more restorative sleep phases, providing a better sleep quality, and helped to regain vigilance (shorter reaction time). The latter behavioural result may lead to an increase of efficiency and productivity after the nap.

While the invention has been disclosed with reference to certain preferred embodiments, numerous modifications, alterations, and changes to the described embodiments, and equivalents thereof, are possible without departing from the scope of the claims. Accordingly, it is intended that the invention not be limited to the described embodiments, and be given the broadest reasonable interpretation in accordance with the language of the appended claims.

1-23. (canceled)

24. A system to accelerate sleep onset and/or to improve sleep quality in a subject, the system comprising:
a haptic device comprising a plurality of tactile displays configured to provide thermo-tactile stimuli to a user;
at least one audio/video device configured to reproduce an audio file, with or without a video file; and
a data processing apparatus operatively connected with said haptic device, and said audio/video device, the data processing apparatus having a processor comprising instructions configured to operate the system to:
reproduce the audio file, with or without a video file, via the audio/video device; and
provide the thermo-tactile stimuli to a distal portion of the subject to increase the degree of dilation of blood vessels in the skin of said distal portion, wherein the thermo-tactile stimuli are provided through the haptic device according to a haptic profile obtained by said audio file, thereby inducing a relaxation state together with thermal regulation in said subject to accelerate sleep onset and/or to improve sleep quality.

25. The system of claim 24, further comprising sensors configured to measure the temperature of the distal portion of the subject, including at least one of a hand, a foot, an ankle, a wrist, head and neck, or variations thereof.

26. The system of claim 24 wherein the plurality tactile displays are flexible for adaptation to the distal portion, and includes a plurality of cells separately controlled in pressure and/or temperature and/or activation time, and/or duration time, via a pressure and/or a temperature of a medium in the plurality of cells.

27. The system of claim 26 wherein, when the plurality of cells are arranged on the distal portion to define a cell pattern.

28. The system of claim 27, further configured to:
determine a wave profile of frequencies and/or amplitudes of an audio signal of said audio file, including peaks and valleys;
identify a plurality of wave parameters in the wave profile, the plurality of wave parameters at least including a rise time and a fall time of the wave profile, a rise duration and a fall duration of the wave profile, a wave slope and/or a wave amplitude of the wave profile, and transform the wave parameters into haptic profile parameters, including an activation time, a activation duration, a pressure and/or a temperature of the cells; and
transmit pluralities of signals to the plurality of cells, each plurality of signals being associated to said haptic profile parameters and being directed to one of said plurality of cells,
wherein the system is configured to transmit the signals activating a first cell, which is arranged in the cell pattern before a second cell, before transmission of the signals for activating the second cell in the pattern.

29. The system of claim 28, configured to begin transmission of the plurality of signals directed to one cell before an end of transmission of other signals to another cell.

30. The system of claim 28, configured to transmit signals directed to one cell at the same time when signals directed to another cell are transmitted.

31. The system of claim 28 wherein duration of the thermo-tactile stimuli is set corresponding to duration of the signals, wherein duration of signals directed to one cell is different or equal to duration of other signals to another cell of the plurality of cells, and wherein a pressure and/or temperature of the thermo-tactile stimuli is set based on body parameters measured in a body portion different from the distal portion.

32. The system of claim 24, further configured to monitor over time the temperature and/or the degree of dilation of blood vessels in the skin of the distal portion of the subject, or variations thereof.

33. The system of claim 24, wherein said audio file comprises at least an audio track encoding a voice and an audio track encoding a sound.

34. The system of claim 24, configured to obtain the haptic profile by:
processing an audio signal derived from the audio file, thereby obtaining at least one profile of frequencies and/or amplitudes of said audio signal; and
converting said frequencies and/or amplitudes profiles into the haptic profile.

35. The system of claim 24 wherein the thermo-tactile stimuli comprise thermal and pressure gradients provided on the subject skin in a spatio-temporal fashion.

36. The system of claim 24, configured to set the thermo-tactile stimuli duration based on physiological, movement and/or psychological parameters of the subject.

37. A non-transitory computer readable medium containing a set of instructions that, when executed by a data processing apparatus comprised in the system of claim 24, cause the data processing apparatus to operate said system to:
reproduce an audio file, with or without a video file, via the audio/video device; and
provide a thermo-tactile stimuli to a distal portion of the subject to increase the degree of dilation of blood vessels in the skin of said distal portion, wherein the thermo-tactile stimuli are provided through the haptic device according to a haptic profile obtained by said audio file, thereby inducing a relaxation state together with thermal regulation in said subject to accelerate sleep onset and/or to improve sleep quality.

38. The non-transitory computer readable medium of claim 37, wherein said instructions comprise:
receiving and/or processing data regarding the temperature of at least one distal portion of the subject, or variations thereof;
operating the audio/video device to reproduce the audio file, with or without the video file;
receiving and/or processing data regarding the haptic profile obtained by said audio file; and
operating the haptic device to provide thermo-tactile stimuli to the distal portion of the subject.
